(19) United States
(12) Patent Application Publication
(10) Pub. No.: US 2005/0059741 A1
(43) Pub. Date: Mar. 17, 2005

(54) Compositions and methods involving the combination of a thromboxane A2 receptor antagonist and an inhibitor of cyclooxygenase-1

(75) Inventor: Hans R. Brunner, Riehen (CH)

Correspondence Address: FITCH, EVEN, TABIN & FLANNERY, P. O. BOX 65973, WASHINGTON, DC, 20035, US

(73) Assignee: B.M.R.A. Corporation B.V.

(21) Appl. No.: 10/911,644

(22) Filed: Aug. 5, 2004

Related U.S. Application Data
(60) Provisional application No. 60/492,975, filed on Aug. 7, 2003.

(51) Int. Cl. A61K 31/557; A61K 31/192

(52) U.S. Cl. 514/570; 514/573

(57) ABSTRACT

The invention is directed to methods and compositions that can be used in the treatment of inflammation, pain, and cardiovascular disorders. Methods and compositions are described involving the combination of a thromboxane A2 receptor antagonist and an inhibitor of cyclooxygenase-1.

### CROSS REFERENCE TO RELATED APPLICATIONS

The present application claims the benefit of U.S. provisional application No. 60/492,975, filed on Aug. 7, 2003, which is incorporated in its entirety herein by reference.

### FIELD OF THE INVENTION

The invention is directed to compositions containing both a cyclooxygenase-1 (COX-1) inhibitor and a thromboxane A2 receptor antagonist. The compositions may be used to treat patients for a variety of cardiovascular conditions, pain and inflammation. The invention also includes methods by which patients are treated.

### BACKGROUND OF THE INVENTION

NSAIDs and Other Cyclooxygenase-1 Inhibitors

NSAIDs are among the most commonly taken drugs for pain and inflammation. The chronic use of one NSAID, aspirin, has also been associated a reduced incidence of cardiovascular disease and many people presently take low doses of aspirin on a daily basis to reduce their risk of stroke and thromboembolism. Aspirin may exert this effect by inhibiting cyclooxygenase-1 (COX-1), an enzyme that contributes to the production of thromboxane. To varying degrees, other NSAIDs also inhibit COX-1 and a second cyclooxygenase (COX-2) (Vane, et al., Am. J. Med. 104:2S-8S (1998); Griswold, et al., Med. Res. Rev. 16(2). 181-206 (1996); Lane, J. Rheumatol 24 (Suppl 49):20-4 (1997); Lipsky, et al., J. Rheumatol. 24(Suppl 49):9-14 (1997)). Because it is widely believed that inhibition of COX-1 is associated with the gastrointestinal side effects of NSAIDs, compounds with a high degree of specificity for COX-2 have been developed and some are now on the market.

More recent research has led many to reconsider the wisdom of blocking one cyclooxygenase enzyme but not the other (Mukherjee, et al., JAMA 286:954-959 (2001); Science 296:539-541 (2002)). COX-2 promotes the production of prostacyclin which dilates blood vessels and prevents platelets from clumping together. The loss of this activity coupled with the continued induction of thromboxane production by COX-1 may significantly increase the risk of adverse cardiovascular events.

Thromboxane A2 Receptor Antagonists

Thromboxane A2/prostaglandin H2 receptor antagonists have been reported to be effective in treating, inter alia, arterial or venous thrombosis, unstable angina, transient ischemic attacks, and hypertension, (U.S. Pat. No. 5,100,889). They include 7-oxabicycloheptane substituted prostaglandin analogs (U.S. Pat. No. 5,100,889; Rosenfeld, et al., Cardiovascular Drug Rev.19:97-115 (2001)), benzenealkonic acids (U.S. Pat. No. 5,618,941), and benzenesulfonamide derivatives (U.S. Pat. No. 5,597,848). In addition, published PCT application WO 99/45905 suggests the use of thromboxane synthetase inhibitors and receptor antagonists for treating conditions such as migraine headache. It also suggests that these compounds may be used in combination with a variety of other agents, including NSAIDs.

### SUMMARY OF THE INVENTION

The present invention is based upon the concept that thromboxane receptor antagonists and COX-1 inhibitors (e.g., NSAIDs that act on COX-1) have complementary activities and may be combined to increase their effectiveness. Thus, the dosages of these compounds, when administered in combination, may be decreased below the dosages normally used in the art and, as a result, the risk of unwanted side effects can be reduced. For example, lower dosages of COX-1 inhibitors should reduce the gastrointestinal side effects associated with these drugs.

Thromboxane receptor antagonists block activity at the level of receptor binding whereas COX-1 inhibitors block the production of thromboxane. These synergistic effects lead to a composition that is more effective at preventing a range of diseases and conditions including stroke and peripheral arterial disease (e.g., atherosclerosis). The combination should also result in an NSAID product that is safer in terms of cardiovascular risk. This is particularly true of NSAIDs that exert a strong effect on COX-2 or which are relatively specific in inhibiting COX-2. The invention includes compositions, therapeutic packages and treatment methods.

In its first aspect, the invention is directed to a pharmaceutical composition in unit dose form which contains a COX-1 inhibitor and a thromboxane A2 receptor antagonist. These drugs are present in an amount that is therapeutically effective upon the administration of one or more unit doses of the composition to a patient but in which one or both of the drugs is present at a lower dosage than that which is accepted to be therapeutically effective when used alone. The term “unit dose” or “unit dosage form” refers to a single drug administration entity. By way of example, a single tablet, capsule, dragee, vial for injection or syringe combining both a COX-1 inhibitor and a thromboxane A2 receptor antagonist would be a unit dose form. As used herein, the term “COX-1 inhibitor” refers to agents that inhibit COX-1 and which may or may not also inhibit COX-2. However, the term does not include COX-2 specific inhibitors. As used herein a “COX-2 specific inhibitor” is a compound that, at a dosage that causes a 50% inhibition of COX-2, inhibits COX-1 by less than 10%. The term “therapeutically effective” means that sufficient drug is present to generate a therapeutic action for which the drug is given. For example, if a patient is being treated for pain, then a “therapeutically effective” amount of NSAID would be a dosage sufficient to reduce the severity or duration of the pain. If the patient is being treated for inflammation, then enough drug would need to be present to reduce the associated pain or swelling. Similarly, if a patient is being administered drug to reduce the risk of developing a cardiovascular problem, then a sufficient amount should be given to accomplish this reduction.

Preferred COX-1 inhibitors are NSAIDs, particularly indobufen (preferably 20-2000 mg/unit dose and more preferably 40-500 mg/unit dose; 40-6000 mg/day); flurbiprofen (particularly at 2-200 mg/unit dose, 2-600 mg/day); naproxen (particularly at 50-600 mg/unit dose, 50-3000 mg/day); oxaprozin (particularly at 40-600 mg/unit dose and 40-1800 mg/day); indomethacin (particularly at 1-100 mg/unit dose, 1-500 mg per day); ketorolac (particularly at 1-100 mg/unit dose, 1-500 mg per day); mefenamic acid (particularly at 50-1000 mg/unit dose, 50-3000 mg per day); nabumetone (particularly at 50-1000 mg per dose, 50-3000 mg per day); and etodolac (particularly at 5-600 mg per unit dose and 5-1800 mg per day). All dosages used herein are based upon administration to a patient, which for the purposes of the present invention is confined to a human. The most preferred COX-1 inhibitor is indobufen. Although aspirin may be used in compositions, it is generally preferred that COX-1 inhibitors other than aspirin be used to avoid the gastrointestinal and other problems associated with this agent.

Thromboxane A2 receptor antagonists that may be used in the compositions include 7-oxabicycloheptane substituted prostaglandin analogs such as those described in U.S. Pat. No. 5,100,889, benzenealkonic acids and benzenesulfonamide derivatives, typically at 1-1000 mg per unit dose and 1-5000 mg per day. The most preferred thromboxane receptor antagonist is ifetroban, particularly at 5-500 mg. It will be understood that, unless otherwise indicated, reference to a COX-1 inhibitor or thromboxane A2 receptor antagonist includes all pharmaceutically acceptable forms of the drug known in the art. For example, any pharmaceutically acceptable salt of a drug may be used in compositions. The weights recited however, refer to the drug itself, for example in its acid or base form.

As mentioned above, since the COX-1 inhibitor and thromboxane receptor antagonist act at two different levels to reduce the effects of thromboxane, a lower individual dosage (i.e., the amount of agent taken at a single time) and daily dosage may be used to accomplish a therapeutic objective than would be required if either drug were used alone. Similarly, unit dosage forms can contain a lower amount of the COX-1 inhibitor and thromboxane A2 receptor antagonist than would be needed if either drug was as the sole active agent. For the purposes of the present invention these lowered dosages are referred to as a “synergistic dosage,” “synergistic daily dosage” and “synergistic unit dosage amount” respectively. Expressed more precisely, a synergistic dosage of a compound is a therapeutically effective dosage that is lower than the minimum effective dose of the same compound (i.e., the lowest amount that can be given to a patient and still obtain a beneficial therapeutic effect) when it is the sole active agent administered. For example, if the lowest therapeutically effective amount of a drug that can be present in a unit dosage form is 40 mg when the drug is the sole active ingredient, then a synergistic unit dosage would be any dosage of the drug lower than 40 mg that is therapeutically effective.

Specific examples are: indobufen in a unit dosage form at less than 50 mg (e.g., 5-45 mg, 5-40 mg or 5-30 mg), given in an individual dose of less than 100 mg (e.g., 5-90 mg) and possibly less than 50 mg (e.g., 5-45 mg) and at a daily dose of less than 200 mg per day (e.g., 5-190 mg or 10-140 mg; or 20-90 mg); flurbiprofen in a unit dosage form at less than 50 mg (e.g., 2-45 mg, 5-40 mg or 5-30 mg), given in a individual dosage of less than 50 mg (e.g., 2-45 mg) and given at a daily dose of less than 100 mg per day (e.g., 5-90 mg or 10-75 mg; or 10-40 mg); naproxen in a unit dosage form at less than 200 mg (e.g., 10-190 mg, 20-140 mg or 30-90 mg), given in an individual dosage of less than 200 mg (e.g 10-90 mg), and given at a daily dose of less than 200 mg per day (e.g., 5-175 mg or 10-140 mg; or 20-90 mg); oxaprozin in a unit dosage form at less than 200 mg (e.g., 5-190 mg, 10-140 mg or 20-90 mg), given in an individual dosage of less than 200 mg (e.g, 5-190 mg) and given at a daily dose of less than 200 mg per day (e.g., 5-175 mg or 10-150 mg; or 20-90 mg); indomethacin in a unit dosage form at less than 25 mg (e.g., 1-20 mg, 2-15 mg or 1-10 mg), given in an individual dosage of less than 25 mg (e.g., 1-20 mg) and given at a daily dose of less than 75 mg per day (e.g., 1-70 mg or 1-60 mg; or 2-50 mg); ketorolac in a unit dosage form at less than 10 mg (e.g., 0.1-9 mg, 0.1-5 mg or 0.5-5 mg), given in an individual dosage of less than 10 mg (e.g., 0.1-9 mg) and given at a daily dose of less than 10 mg per day (e.g., 0.1-9 mg or 0.5-7 mg; or 0.5-5 mg); mefenamic acid in a unit dosage form at less than 100 mg (e.g., 1-90 mg, 2-40 mg or 5-30 mg), given in an individual dosage of less than 100 mg (e.g. 1-90 mg) and given at a daily dose of less than 100 mg per day (e.g., 2-90 mg or 5-75 mg; or 5-40 mg); nabumetone in a unit dosage form at less than 500 mg (e.g., 5-450 mg, 50-390 mg or 50-190 mg), given in an individual dosage of less than 500 mg (e.g., 5-450 mg) and given at a daily dose of less than 1000 mg per day (e.g., 50-900 mg or 50-490 mg; or 50-190 mg); and etodolac in a unit dosage form at less than 200 mg (e.g., 2-190 mg, 2-140 mg or 5-90 mg), given in an individual dosage of less than 200 mg (e.g., 2-190 mg) and given at a daily dose of less than 600 mg per day (e.g., 50-590 mg or 50-390 mg; or 50-190 mg). In each case amounts are based upon the assumption of a solid oral dosage form such as a tablet or capsule.

Synergistic dosages, synergistic daily dosages and synergistic unit dosage amounts of thromboxane A2 receptor antagonists may also be used. In accordance with the definitions discussed above, a synergistic amount is an amount of thromboxane A2 receptor antagonist sufficient to accomplish a therapeutic objective (in combination with a COX-1 inhibitor) that is lower than the minimum effective amount of thromboxane receptor antagonist when it is used alone. An antagonist, e.g., ifetroban, may be present in combination at less than 200 mg (e.g., 10-190 mg, 10-140 mg, or 10-90 mg), and administered as either an individual dose or total daily dose of less than 1 mg per kg body weight (e.g., 0.5-0.9 mg/kg, 0.5-0.7 mg/kg). The most preferred composition in this respect is a unit dosage form, preferably a tablet or capsule for oral administration, having indobufen and ifetroban with either or both present in a synergistic amount. Similarly, preferred treatment methods involve the co-timely administration of indobufen and ifetroban with either or both given at a synergistic dosage or synergistic daily dosage. The specific therapeutic objectives referred to above may be any of the positive effects that have been attributed to the administration of a COX-1 inhibitor or a thromboxane A2 receptor inhibitor. Particular therapeutic objectives are: reducing the risk of arterial or venous thrombosis, reducing the incidence and severity of unstable angina, reducing the incidence or severity of transient ischemic attacks, reducing hypertension and reducing the number or severity of atherosclerotic lesions.

The therapeutic agents described above, i.e., the COX-1 inhibitor and the thromboxane A2 receptor antagonist, may be supplied in the form of a therapeutic package. Each package has one or more finished pharmaceutical containers with the therapeutic agents in unit dose form and includes labeling directed to their use in the treatment or prevention of a condition responsive to a COX-1 inhibitor or a thromboxane A2 receptor antagonist. Preferred conditions include cardiovascular conditions (e.g., arterial or venous thrombosis, peripheral arterial disease, angina, or hypertension). The COX-1 inhibitor, the thromboxane A2 receptor antagonist or both may be present in the one or more unit doses in a synergistic unit dosage amount

The invention also includes methods of treating a patient for, or to prevent, a condition responsive to a COX-1 inhibitor or a thromboxane A2 receptor antagonist by either administering the pharmaceutical compositions described above or by sequentially administering the two drugs in a co-timely manner, ie., the second drug is administered while the first drug is still present an amount that, upon administration of the second drug, is therapeutically effective. Preferably one or both of the drugs are administered at a synergistic dosage Any of the specific conditions mentioned above may be treated in this manner. The preferred agents are ifetroban and indobufen.

### DETAILED DESCRIPTION OF THE INVENTION

A. COX-1 Inhibitors and Thromboxane A2 Receptor Antagonists

All of the COX-1 inhibitors described herein have been well known in the art for many years and may be either purchased commercially or synthesized using standard methods. Similarly, a variety of thromboxane A2 receptor antagonists have been disclosed and methods for synthesizing these compounds have been described for bicycloheptane substituted prostaglandin analogs (U.S. Pat. No. 5,100,889; Rosenfeld, et al., Cardiovascular Drug Rev. 97-115 (2001)), benzenealkonic acids (U.S. Pat. No. 5,618,941), and benzenesulfonamide derivatives (U.S. Pat. No. 5,597,848). Any of these prior methods may be used to obtain agents suitable for use in the present invention.

B. Route of Administration

The methods and compositions discussed above are compatible with any dosage form or route of administration. Thus, agents may be administered orally, intranasally, rectally, sublingually, buccally, parenterally, or transdermally. Dosage forms may include tablets, trochees, capsules, caplets, dragees, lozenges, parenterals, liquids, powders, and formulations designed for implantation or administration to the surface of the skin. The most preferred dosage forms are tablets or capsules for oral administration. All dosage forms may be prepared using methods that are standard in the art (see e.g., Remington's Pharmaceutical Sciences, 16th ed. A. Oslo. ed., Easton, Pa. (1980)).

Active ingredients may be used in conjunction with any of the vehicles and excipients commonly employed in pharmaceutical compositions, e.g., talc, gum arabic, lactose, starch, magnesium stearate, cocoa butter, aqueous or non-aqueous solvents, oils, paraffin derivatives, glycols, etc. Coloring and flavoring agents may also be added to preparations designed for oral administration. Solutions can be prepared using water or physiologically compatible organic solvents such as ethanol, 1-2 propylene glycol, polyglycols, dimethyl sulfoxide, fatty alcohols, triglycerides, partial esters of glycerin, and the like. Parenteral compositions containing active ingredients may be prepared using conventional techniques and include sterile isotonic saline, water, 1,3-butanediol, ethanol, 1,2-propylene glycol, polyglycols mixed with water, Ringer's solution, etc.

C. Treatment Methods

The combinations of COX-1 inhibitors and thromboxane receptor antagonists described herein are especially useful in the treatment and prevention of peripheral arterial disease, arterial or venous thrombosis, unstable angina, transient ischemic attacks and hypertension. Thus, the invention includes methods of treating these conditions by administering a thromboxane A2 receptor antagonist in combination with a COX-1 inhibitor. These agents should be given in a co-timely manner and should be delivered in an amount sufficient to reduce the symptoms of the underlying disease. Preferably the agents are delivered together in a unit dosage form as described herein.

D. Dosages

With respect to therapeutic agents, it is expected that the skilled practitioner will adjust dosages on a case by case basis using methods well established in clinical medicine. General guidance concerning appropriate amounts of a variety of agents is provided above. However, these are simply guidelines since the actual dose will be carefully selected and titrated by the attending physician based upon clinical factors unique to each patient. The optimal daily dose will be determined by methods known in the art and will be influenced by factors such as the age of the patient, the disease state, side effects associated with the particular agent being administered and other clinically relevant factors. In some cases, a patient may already be taking medications at the time that treatment with the present combination is initiated. These other medications may be continued provided that no unacceptable adverse side effects are reported by the patient. Daily dosages may be provided in either a single or multiple regimen with the latter being generally preferred.

All references cited herein are fully incorporated by reference. Having now fully described the invention. It will be understood by one of skill in the art that the invention may be performed within a wide and equivalent range of conditions, parameters and the like, without affecting the spirit or scope of the invention or any embodiment thereof.

1. A pharmaceutical composition in unit dose form, comprising:
(a) a COX-1 inhibitor; and
(b) a thromboxane A2 receptor antagonist;
wherein said COX-1 inhibitor, said thromboxane A2 receptor antagonist or both are present in a synergistic unit dosage amount.

2. The pharmaceutical composition of claim 1, wherein said COX-1 inhibitor is an NSAID other than aspirin.

3. The pharmaceutical composition of claim 2, wherein said NSAID is selected from the group consisting of: indobufen; flurbiprofen; naproxen; oxaprozin; indomethacin; ketorolac; mefenamic acid; nabumetone; and etodolac.

4. The pharmaceutical composition of claim 3, wherein said NSAID is indobufen.

5. The pharmaceutical composition of claim 4, wherein said indobufen is present in an amount of less than 50 mg.

6. The pharmaceutical composition of any one of claims 1-5, wherein said thromboxane A2 receptor antagonist is a 7-oxabicycloheptane substituted prostaglandin analog; a benzenealkonic acid; or a benzenesulfonamide derivative.

7. The pharmaceutical composition of claim 6, wherein said thromboxane A2 receptor antagonist is a 7-oxabicycloheptane substituted prostaglandin analog.

8. The pharmaceutical composition of claim 7, wherein said 7-oxabicycloheptane substituted prostaglandin analog is ifetroban.

9. The pharmaceutical composition of claim 8, wherein said ifetroban is present in an amount of less than 200 mg.

10. The pharmaceutical composition of claim 6, wherein said COX-1 inhibitor is indobufen in an amount of less than 50 mg and said thromboxane A2 receptor antagonist is ifetroban in an amount of less than 200 mg.

11. The pharmaceutical composition of claim 10, wherein said pharmaceutical composition is in the form of a tablet or capsule for oral administration.

12. A therapeutic package for dispensing to a patient which comprises:
(a) one or more unit doses, each such unit dose comprising:
(i) a COX-1 inhibitor; and
(ii) a thromboxane A2 receptor antagonist;
wherein said COX-1 inhibitor and said thromboxane A2 receptor antagonist are present in an amount sufficient to be therapeutically effective upon the administration of one or more of said unit doses to a patient; and
(b) a finished pharmaceutical container therefore, said container enclosing said unit dose or unit doses, and further comprising labeling directed to the use of said one or more unit doses in the treatment or prevention of a condition selected from the group consisting of: a cardiovascular condition; peripheral arterial disease; arterial or venous thrombosis; angina; transient ischemic attack; stroke; and hypertension.

13. The therapeutic package of claim 12, wherein said labeling is directed to the use of said one or more unit doses in the treatment or prevention of a cardiovascular condition.

14. The therapeutic package of claim 12, wherein said labeling is directed to the use of said one or more unit doses in the treatment or prevention of a condition selected from the group consisting of: peripheral arterial disease; and arterial or venous thrombosis.

15. The therapeutic package of claim 12, wherein said labeling is directed to the use of said one or more unit doses in the treatment or prevention of a condition selected from the group consisting of: stroke; and hypertension.

16. A therapeutic package for dispensing to a patient which comprises:
(a) one or more unit doses, each such unit dose comprising:
(i) a COX-1 inhibitor; and
(ii) a thromboxane A2 receptor antagonist;
wherein said COX-1 inhibitor, said thromboxane A2 receptor antagonist or both are present in said one or more unit doses in a synergistic unit dosage amount; and
(b) a finished pharmaceutical container therefore, said container enclosing said unit dose or unit doses, and further comprising labeling directed to the use of said one or more unit doses in the treatment or prevention of a condition responsive to a COX-1 inhibitor or a thromboxane A2 receptor antagonist.

17. The therapeutic package of any one of claims 12-16, wherein said COX-1 inhibitor is an NSAID other than aspirin.

18. The therapeutic package of claim 17, wherein said NSAID is selected from the group consisting of: indobufen; flurbiprofen; naproxen; oxaprozin; indomethacin; ketorolac; mefenamic acid; nabumetone; and etodolac.

19. The therapeutic package of claim 18, wherein said NSAID is indobufen present in each of said one or more unit dosage forms in an amount of less than 50 mg.

20. The therapeutic package of claim 18, wherein said thromboxane A2 receptor antagonist is a 7-oxabicycloheptane substituted prostaglandin analog; a benzenealkonic acid; or a benzenesulfonamide derivative.

21. The therapeutic package of claim 20, wherein said thromboxane A2 receptor antagonist is a 7-oxabicycloheptane substituted prostaglandin analog.

22. The therapeutic package of claim 21, wherein said 7-oxabicycloheptane substituted prostaglandin analog is ifetroban and is present in each of said one or more unit dosage forms in an amount of less than 200 mg.

23. The therapeutic package of any one of claims 12-16, wherein said thromboxane A2 receptor antagonist is a 7-oxabicycloheptane substituted prostaglandin analog; a benzenealkonic acid; or a benzenesulfonamide derivative.

24. The therapeutic package of claim 23, wherein said thromboxane A2 receptor antagonist is ifetroban.

25. The therapeutic package of any one of claims 12-16, wherein said COX-1 inhibitor is indobufen present in each of said one or more unit dosage forms in an amount of less than 50 mg and said thromboxane A2 receptor antagonist is ifetroban present in each of said one or more unit dosage forms in an amount of less than 200 mg.

26. A method of treating or preventing a disease or condition in a patient wherein said disease or condition is responsive to either a COX-1 inhibitor or a thromboxane A2 receptor antagonist, comprising administering to said patient the pharmaceutical composition of any one of claims 1-5.

27. A method of treating or preventing a disease or condition in a patient which is responsive to a COX-1 inhibitor comprising co-timely administering to said patient:
(a) a COX-1 inhibitor at a synergistic dosage; and
(b) a thromboxane A2 receptor antagonist at a dosage which, together with the synergistic dosage of COX-1 inhibitor of paragraph (a), is effective in the prevention or treatment of said disease or condition.

28. The method of claim 26, wherein said disease or condition is inflammation or pain.

29. The method of claim 26 wherein said COX-1 inhibitor is an NSAID other than aspirin.

30. The method of claim 29, wherein said NSAID is selected from the group consisting of: indobufen; flurbiprofen; naproxen; oxaprozin; indomethacin; ketorolac; mefenamic acid; nabumetone; and etodolac.

31. The method of claim 30, wherein said NSAID is indobufen at a dosage of less than 100 mg.

32. The method of claim 30, wherein said thromboxane A2 receptor antagonist is a 7-oxabicycloheptane substituted prostaglandin analog; a benzenealkonic acid; or a benzenesulfonamide derivative.

33. The method of claim 30, wherein said thromboxane A2 receptor antagonist is administered at a dosage of 1-1000 mg.

34. The method of claim 27-31, wherein said thromboxane A2 receptor antagonist is ifetroban at 5-500 mg.

35. A method of treating or preventing a disease or condition in a patient which is responsive to a thromboxane A2 receptor antagonist, comprising co-timely administering to said patient:
(a) a thromboxane A2 receptor antagonist at a synergistic dosage; and
(b) a COX-1 inhibitor at a dosage which, together with the synergistic dosage of thromboxane A2 antagonist of paragraph (a), is effective in the prevention or treatment of said disease or condition.

36. The method of claim 35, wherein said disease or condition is selected from the group consisting of: stroke; arterial or venous thrombosis; unstable angina; a transient ischemic attack; and hypertension,

37. The method of claim 36 wherein said COX-1 inhibitor is an NSAID other than aspirin.

38. The method of claim 37, wherein said NSAID is selected from the group consisting of: indobufen; flurbiprofen; naproxen; oxaprozin; indomethacin; ketorolac; mefenamic acid; nabumetone; and etodolac.

39. The method of claim 36, wherein said thromboxane A2 receptor antagonist is a 7-oxabicycloheptane substituted prostaglandin analog; a benzenealkonic acid; or a benzenesulfonamide derivative.

40. The method of claim 35-39, wherein said thromboxane A2 receptor antagonist is ifetroban administered to said patient at a dosage of less than 1.0 mg per kg body weight.

41. A method of treating a patient for a condition selected from the group consisting of: a cardiovascular condition; peripheral arterial disease; arterial or venous thrombosis; angina; transient ischemic attack; stroke; and hypertension comprising: administering to said patient in a co-timely manner:
(a) a COX-1 inhibitor; and
(b) a thromboxane A2 receptor antagonist;
wherein said COX-1 inhibitor and said thromboxane A2 receptor antagonist are administered in a therapeutically effective amount.

42. The method of claim 41, wherein said disease or condition is a cardiovascular disease or condition.

43. The method of claim 41, wherein said disease or condition is selected from the group consisting of: peripheral arterial disease; and arterial or venous thrombosis.

44. The method of claim 41, wherein said disease or condition is selected from the group consisting of: stroke; and hypertension.

45. The method of any one of claims 41-44, wherein said COX-1 inhibitor is an NSAID other than aspirin.

46. The method of claim 45, wherein said NSAID is selected from the group consisting of: indobufen; flurbiprofen; naproxen; oxaprozin; indomethacin; ketorolac; mefenamic acid; nabumetone; and etodolac.

47. The method of claim 46, wherein said NSAID is indobufen administered to said patient at a dose of 40-6000 mg per day.

48. The method of claim 46, wherein said thromboxane A2 receptor is ifetroban administered to said patient at a dose of 5-5000 mg per day.

49. The method of any one of claims 41-44, wherein said thromboxane A2 receptor antagonist is a 7-oxabicycloheptane substituted prostaglandin analog; a benzenealkonic acid; or a benzenesulfonamide derivative.

50. The method of claim 49, wherein said thromboxane A2 receptor antagonist is a 7-oxabicycloheptane substituted prostaglandin analog.

51. The method of claim 50, wherein said 7-oxabicycloheptane substituted prostaglandin analog is ifetroban.

52. The method of any on of claims 41-44, wherein said COX-1 inhibitor is indobufen administered at a dose of 40-6000 mg per day and said thromboxane A2 receptor antagonist is ifetroban administered at a dose of 5-5000 mg per day.
